(19) United States
(12) Patent Application Publication
(10) Pub. No.: US 2025/0352052 A1
(43) Pub. Date: Nov. 20, 2025

(54) HYSTEROSCOPIC SYSTEM

(71) Applicant: Covidien LP, Mansfield, MA (US)

(72) Inventors: Mira Sahney, South Lexington, MA (US); Cemal Shener-Irmakoglu, Hot Springs, AR (US)

(21) Appl. No.: 19/280,425

(22) Filed: Jul. 25, 2025

Related U.S. Application Data
(63) Continuation of application No. 18/393,030, filed on Dec. 21, 2023, now Pat. No. 12,369,788.
Application No. 18/393,030 is a continuation of application No. 17/574,759, filed on Jan. 13, 2022, now Pat. No. 11,889,993.
Application No. 17/574,759 is a division of application No. 16/358,848, filed on Mar. 20, 2019, now Pat. No. 11,229,354.
Application No. 16/358,848 is a division of application No. 14/846,198, filed on Sep. 4, 2015, now Pat. No. 10,251,539.
Application No. 14/846,198 is a continuation of application No. 12/892,355, filed on Sep. 28, 2010, now Pat. No. 9,155,454.

(51) Int. Cl. A61B 1/303; A61B 1/00; A61B 1/015; A61B 1/018; A61B 1/307; A61B 17/32; A61B 17/3205; A61B 17/34; A61B 17/42

(52) U.S. Cl. CPC A61B 1/303; A61B 1/00135; A61B 1/015; A61B 1/018; A61B 17/3421; A61B 17/42; A61B 1/307; A61B 17/32002; A61B 2017/320024; A61B 2017/320028; A61B 2017/320052; A61B 17/3205; A61B 2017/4216; A61B 2217/007

(57) ABSTRACT

A hysteroscopy system includes a scope having an internal channel, a sheath removably coupled to the scope, and an outflow channel. The sheath has a distal flange extending internally towards an outer surface of the scope. The outflow channel is formed between an inner surface of the sheath and an outer surface of the scope. The distal flange forms a distal end of the outflow channel and is generally located between the scope and the sheath.

### CROSS-REFERENCE TO RELATED APPLICATIONS

This application is a continuation of U.S. patent application Ser. No. 18/393,030, filed on Dec. 21, 2023, which is a continuation of U.S. patent application Ser. No. 17/574,759, filed on Jan. 13, 2022 and now U.S. Pat. No. 11,889,993, which is a divisional of U.S. patent application Ser. No. 16/358,848, filed on Mar. 20, 2019 and now U.S. Pat. No. 11,229,354, which is a divisional of U.S. patent application Ser. No. 14/846,198, filed on Sep. 4, 2015 and now U.S. Pat. No. 10,251,539, which is a continuation of U.S. patent application Ser. No. 12/892,355, filed on Sep. 28, 2010 and now U.S. Pat. No. 9,155,454.

### FIELD OF THE INVENTION

The present invention relates generally to hysteroscopy systems, and, more particularly, to a hysteroscopy system having a small size for use in an office setting.

### BACKGROUND OF THE INVENTION

Hysteroscopy refers generally to the inspection of a uterine cavity using a hysteroscope with access through the cervix. As such, hysteroscopy allows diagnosis of intrauterine pathology and, furthermore, can be used for surgical intervention. The hysteroscope typically includes a scope and a sheath.

One problem associated with some current hysteroscopy systems is that they must be used in an operating room setting with the patient being under some type of anesthesia. Anesthesia is required in particular because the size of current hysteroscopes is large and, as such, they can cause discomfort and pain to the patient. For example, a typical hysteroscope may have an outermost diameter of about 9 millimeters. Such hysteroscopes include a scope having a diameter of about 8 millimeters and a sheath having a diameter of about 9 millimeters. In comparison, scientific literature on the subject agrees that hysteroscopy can be performed using a vaginoscopic approach, which can be performed in an office setting, only when the outermost diameter of the hysteroscope is about 6 millimeters or less.

Another problem associated with current scopes is that they typically include a blunt flange at the scope distal end. The flange extends outwardly from the scope and make it difficult, if not impossible, to use the scope without the sheath and/or without an obturator. Accordingly, the size of some current hysteroscopes is limited to the size of the scope and the sheath, e.g., a diameter of 9 millimeters.

What is needed, therefore, is a hysteroscope system for an office setting that addresses the above-stated and other problems.

### SUMMARY

According to one aspect of the present invention, a hysteroscopy system is directed to performing a medical procedure in an office setting. The hysteroscopy system includes a scope having an outer surface, an internal channel defined by an inner surface, and a distal end. A sheath is removably coupled to the scope and has a tip at which a distal flange extends internally towards the outer surface of the scope. The sheath also has an inner surface and a plurality of outflow holes near the distal flange. An outflow channel is formed between the inner surface of the sheath and the outer surface of the scope, the distal flange forming a distal end of the outflow channel. An operative channel is formed within the internal channel of the scope for receiving at least one of a surgical tool and an inflow fluid, and a visualization channel is formed adjacent to the operative channel for receiving a visualization device.

According to yet another aspect of the invention, a hysteroscopy system for a medical procedure includes a scope in the form of an elongated tubular member having an outer surface and an internal surface. The internal surface of the scope defines an internal channel of the scope. A sheath is in the form of an elongated tubular member removably coupled to the scope, the sheath having an outer surface and an internal surface. The sheath has a flange extending internally towards the outer surface of the scope at a distal end of the sheath. An operative member is located within the internal channel of the scope and is in the form of an elongated D-shape tubular member. The operative member has an outer surface and an internal surface, the outer surface being spaced away from the internal surface of the scope to form a visualization channel.

According to yet another aspect of the invention, a hysteroscopy system includes a scope having an outer surface and an internal channel, and a sheath removably coupled to the scope. The sheath has an inner surface and a distal flange, the distal flange extending internally towards the outer surface of the scope. An outflow channel is formed between the inner surface of the sheath and the outer surface of the scope, the distal flange forming a distal end of the outflow channel between the scope and the sheath.

Additional aspects of the invention will be apparent to those of ordinary skill in the art in view of the detailed description of various embodiments, which is made with reference to the drawings, a brief description of which is provided below.

### BRIEF DESCRIPTION OF THE DRAWINGS

FIG. 1 is a perspective view of a hysteroscope system.

FIG. 2A is an enlarged perspective view of a distal end of the hysteroscope system.

FIG. 2B is an enlarged side view of the distal end of the hysteroscope system.

FIG. 2C is an enlarged cross-sectional end view of the distal end of the hysteroscope system.

FIG. 2D is an enlarged cross-sectional top view of the distal end of the hysteroscope system.

FIG. 3 shows dimensions associated with the distal end of FIG. 2A.

FIG. 4 is a perspective view of the hysteroscope system having a sheath removed from a scope.

FIG. 5 is an enlarged perspective view of the distal end of the hysteroscope system of

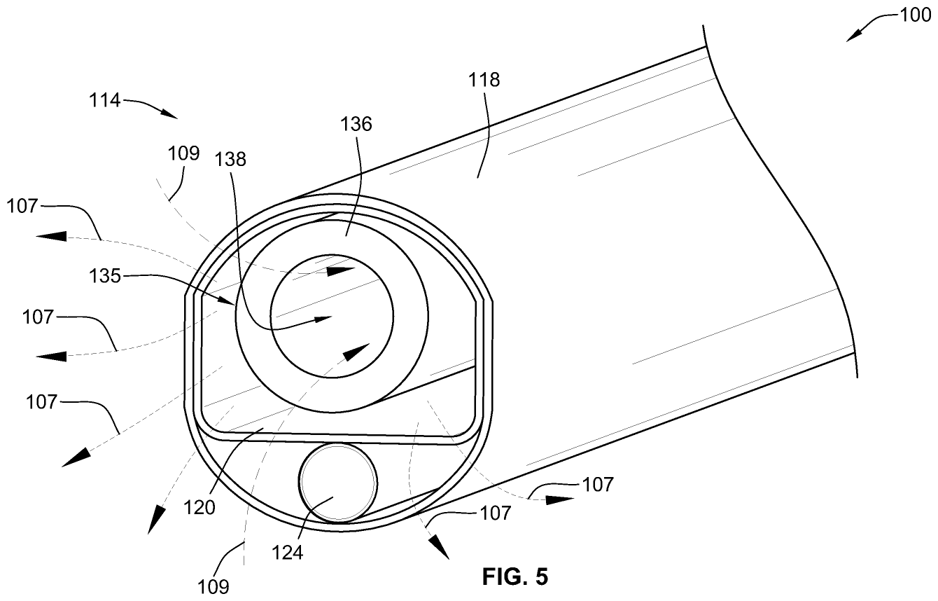

FIG. 4.

FIG. 6 is a perspective view of an alternative embodiment of the hysteroscope system.

While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention as defined by the appended claims.

### DETAILED DESCRIPTION

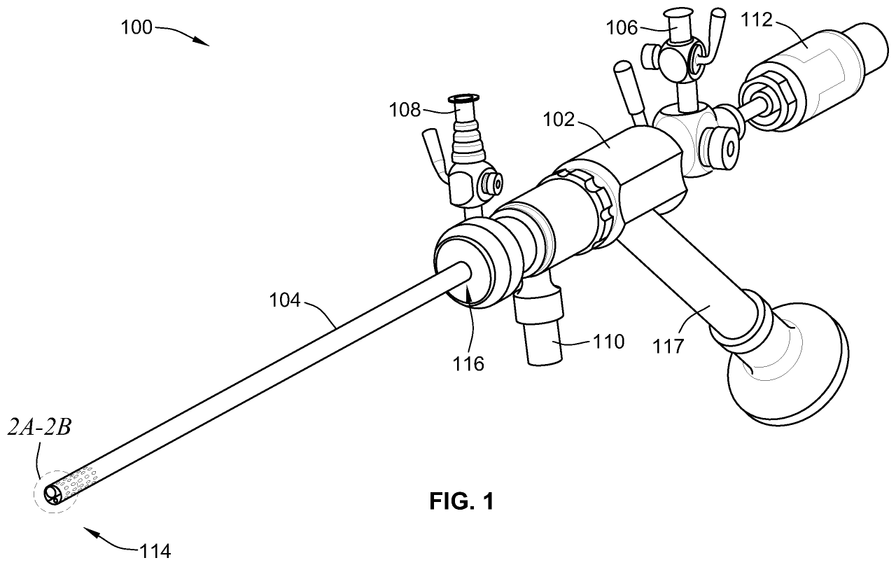

Referring to FIG. 1, a hysteroscope system 100 includes a hysteroscope 102 having, inter alia, a sheath 104, an inflow valve 106, an outflow valve 108, a light post 110, and a morcellator 112. The sheath 104 is a generally an elongated tubular member that has a distal end 114 and a proximal end 116. The hysteroscope system 100 also includes an arm 117 that is connected to an imaging device (e.g., a camera) to capture images received via a visualization device (e.g., visualization device 124).

According to some exemplary embodiments, the hysteroscope system 100 is intended for morcellation of uterine pathology with a scope and accessories having a sufficiently small diameter that can be inserted into a patient's uterus using the vaginoscopic approach. In particular, the hysteroscope system 100 provides a way to minimize patient pain because a tenanculum and speculum are not typically used.

Furthermore, anesthesia is not needed and the medical procedures can be performed in an office setting. This may result, for example, in a quicker surgery with less pain and quicker recovery, and may potentially lower the cost of the surgery. Yet another advantage of the hysteroscope system 100 is that a surgeon has the option to decide if they prefer greater flow instruments (e.g., with a coupled sheath 104) or smaller diameter instruments (e.g., with a removed sheath 104), depending on the patient case.

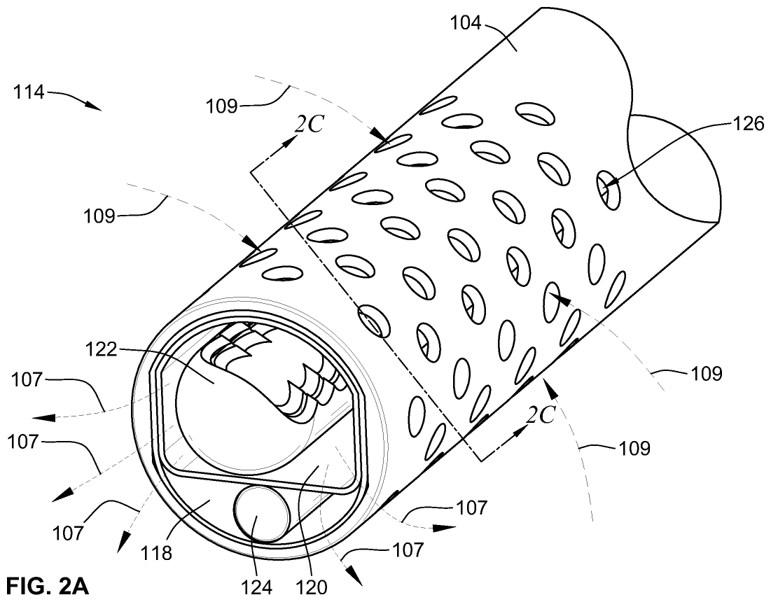

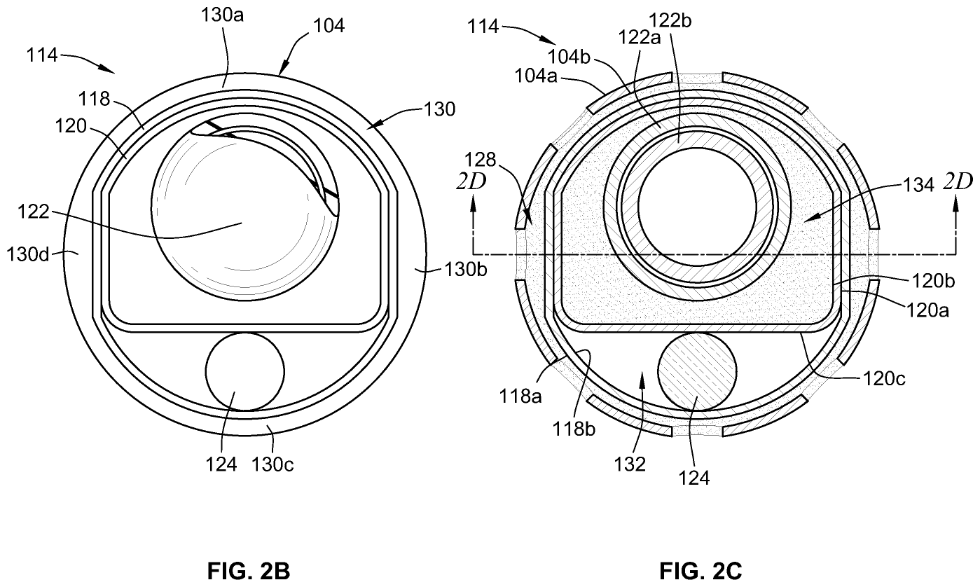

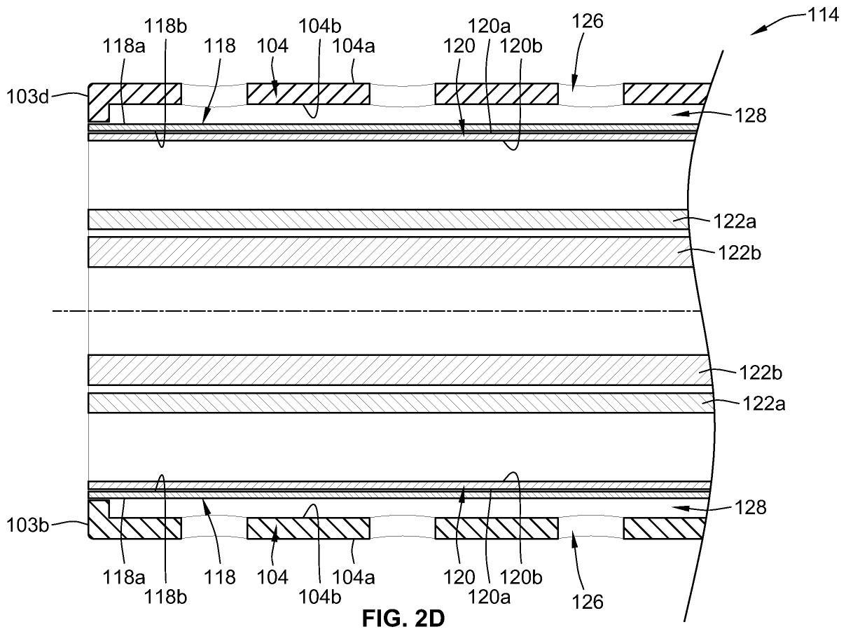

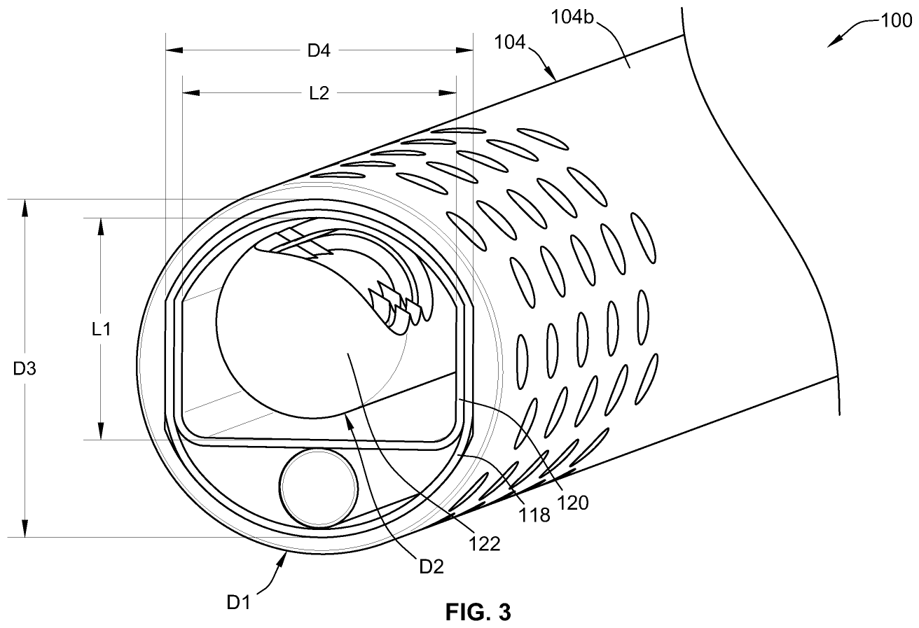

Referring to FIG. 2A, the sheath 104 is removably coupled to a scope 118, which is generally an elongated tubular member having (similar to the sheath 104) a distal end 114 and a proximal end 116. More specifically, the sheath 104 is slidably fitted in an overlapping manner over the scope 118. The scope 118 includes an operative member 120, which is in the form of an elongated D-shape tubular member.

The operative member 120 receives internally a surgical tool 122, which can be selected from a variety of different tools. For example, the surgical tool 122 can be a rotary morcellator, a reciprocating morcellator, or a morcellator having both reciprocal and rotary capabilities. The scope 118 further includes a visualization device 124. The visualization device 124 is adjacent to the operative member 120 and can include various image devices. For example, the visualization device 124 can include fiber-optic technology for illumination and image transmission.

To maintain continuous outflow, a plurality of outflow holes 126 are formed near the distal end 114 of the sheath 104. The inflow valve 106 (shown in FIG. 1) regulates inflow of a liquid through the operative member 120, as represented by the arrows 107 extending from the operative member 120. The liquid is used, for example, to distend and irrigate the uterus of a patient. Furthermore, the liquid is generally received from an access pump, which delivers the fluid to produce a substantially constant predetermined pressure level within a joint cavity, e.g., a uterus. The outflow valve 108 (shown in FIG. 1) regulates outflow of the liquid through the outflow holes 126 via an outflow channel 128 (shown in FIG. 2C) formed between the sheath 104 and the scope 118. The outflow of the liquid is represented by the arrows 109 extending into the outflow holes 126. The outflow liquid is generally sent to a waste container.

Referring to FIG. 2B, the sheath 104 has at the distal end 114 a flange 130 extending inwardly towards the scope 118 to form a closed end of the outflow channel 128 (shown in FIG. 2C). The flange 130 has a generally oval shape and includes two pairs of opposite sides 130a-130d.

According to the illustrated embodiment, the shape of the flange 130 is non-uniform. For example, a second side 130b extends a greater distance internally towards the center of the scope 118 than a first side 130a. Similarly, based on the symmetric features of this embodiment, a fourth side 130d extends a greater distance internally towards the center of the scope 118 than a third side 130c. In alternative embodiments, the flange 130 can have different shapes and sizes.

Referring to FIGS. 2C-2D, the sheath 104 has an outer surface 104a and an internal surface 104b, and the scope 118 has an outer surface 118a and an internal surface 118b. The internal surface 104b of the sheath 104 defines an internal channel in which the scope 118 and the visualization device 124 are located. The internal surface 118b of the scope 118 defines an internal channel in which the outer member and thus the surgical tool 122 is located.

The operative member 120 has an outer surface 120a, an internal surface 120b, and a flat outer surface 120c (clearly shown in FIG. 2C). The flat outer surface 120c is spaced away from the internal surface 118b of the scope 118 to form a visualization channel 132 (clearly shown in FIG. 2C) in which the visualization device 124 is located. The visualization channel 132 is only a small part of the larger internal channel of the scope 118.

The outflow channel 128 is formed between the internal surface 104b of the sheath 104 and the outer surface 118a of the scope 118. An inflow channel 134 is formed in the internal channel of the scope 118. If the surgical tool 122 is removed, the inflow channel 134 is simply the entire internal channel of the scope 118. If the surgical tool 122 is in place, the inflow channel 134 is limited to the area between the surgical tool 122 and the internal surface 120b of the operative member 120.

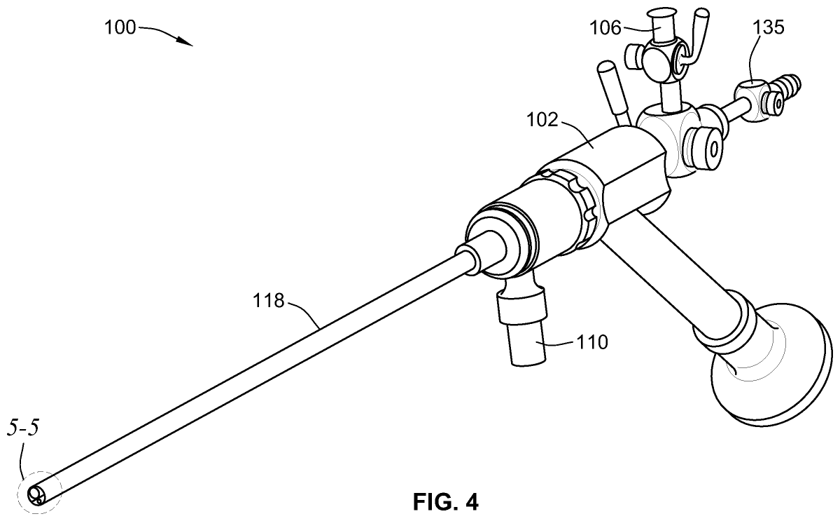

Referring to FIG. 3, the hysteroscopy system 100 is designed to have a size that can be used in an office setting. Specifically, the outer most diameter is designed to be about 6 millimeters or less. According to the illustrated embodiment, the outer diameter Dl of the sheath 104 (which is the same as the diameter of the outer surface 104b) is about 5.6 millimeters. For example, in an alternative embodiment the diameter of the sheath 104 is 5.5 millimeters. The outer diameter D2 of the surgical tool 122 (e.g., morcellator) is about 2.9 millimeters.

The scope 118 has an oval shape with a long diameter D3 of about 5.15 millimeters and a short diameter D4 of about 4.6 millimeters. The operative member 120 has a curvature dimension L1 of about 3.1 millimeters and a flat dimension L2 of about 3.95 millimeters.

The relatively small dimensions of the hysteroscopy system 100 allows a patient to be treated in an office setting. Generally, medical procedures may be provided to a patient with the use of the current hysteroscopy system 100 such that little or no anesthesia may be necessary. Clearly, one advantage of the hysteroscopy system 100 is that it is sufficiently small in diameter to be suitable for the vaginoscopic approach.

Referring to FIG. 4, the hysteroscopy system 100 is also usable without the sheath 104 while still providing continuous flow via a diagnostic cannula 135 (e.g., a cannula having a diameter of about 2.9 millimeters). Specifically, the sheath 104 is removed to allow only the insertion of the scope 118 into a patient, e.g., into a uterus. The removal of the sheath 104 decreases the outermost diameter of the hysteroscopy system 100. For example, in accordance with the dimensions described above in reference to FIG. 3, the outermost diameter decreases to about 5.15 millimeters (the long diameter D3) from about 5.6 millimeters (the outer diameter Dl). When the sheath 104 is removed, the outflow can be provided by an operation tool, such as the morcellator 112 described above (shown in FIGS. 1-3), or by the diagnostic cannula 135.

In contrast to previous scopes, the scope 118 does not have a flange extending outwards from its distal end. The outward extending flange of the previous scopes unnecessarily increased the outermost diameter of the respective scopes and created an obtrusive distal end that made it difficult, if not impossible, to introduce into a patient without a sheath and obturator.

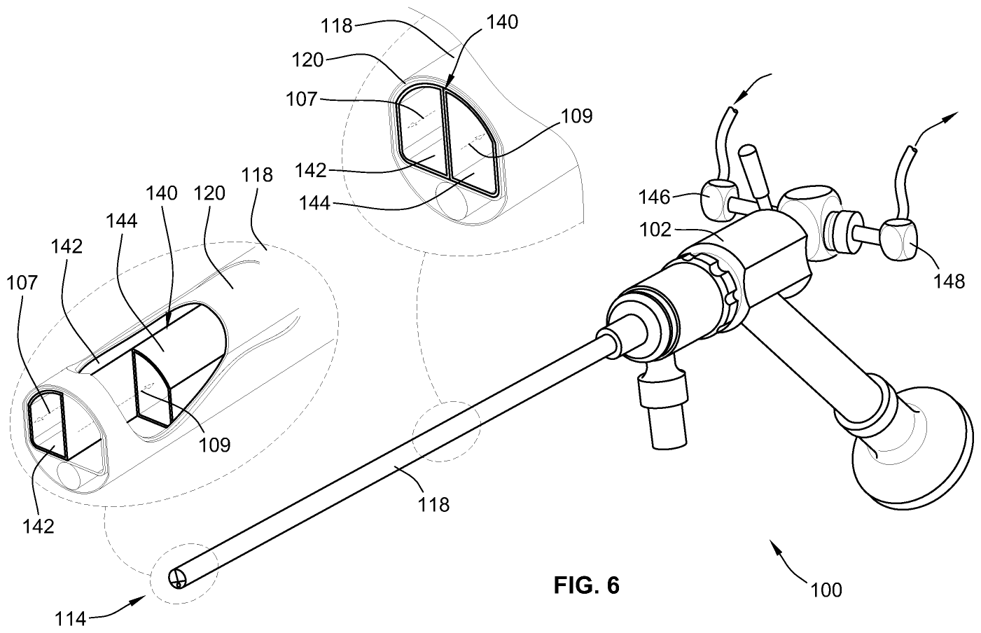

Referring to FIG. 5, the hysteroscopy system can be used for diagnostic purposes when the sheath 104 is removed. The sheath 104 is likely to be used in operative cases mostly to clear the visual field before introduction of a morcellator blade. The diagnostic cannula 135, which has a distal end 136, is used to create a smaller overall diameter of the system for diagnostic purposes. According to the dimensions described above, a reduction of approximately 0.5 millimeters can be achieved by removing the sheath 104. Another advantage of the cannula 135 is that it can be made reusable. Yet another advantage of the cannula 135 is that it can be used to distend and irrigate the patient's uterus during the diagnostic procedure.

The cannula 135 allows for continuous outflow but does not extend beyond the distal end of the scope 118. For example, the cannula 135 provides a replacement for the outflow channel 128, which is removed with the removal of the sheath 104. Specifically, the cannula 135 provides an alternative outflow channel 138 to replace the outflow channel 128 formed by the sheath 104. As such, continuous flow can be maintained even if the sheath 104 is removed.

Referring to FIG. 6, the hysteroscope system 100 alternatively includes a flow device 140 inserted within the operative member 120. The flow device 140 has an inflow tubular element 142 and an outflow tubular element 144, which can be conjoined elements or separate elements.

The distal ends of the tubular elements 142, 144 terminate at different points within the operative member 120. Preferably, the distal end of the inflow tubular element 142 terminates at the distal end 114 of the scope 118, and the distal end of the outflow tubular element 144 terminates some distance away from the distal end 114 within the operative member 120. The termination of tubular elements 142, 144 at different points along the operative member 120 eliminates the possibility of fluid short-circuit and provides better circulation and, hence, irrigation within the uterus.

According to one example, the flow device 140 is made of stainless steel and, as such, can be a reusable device. According to another example, the flow device 140 is made from a much more cost-effective material, such as a polymer. If a polymer is used, the flow device 140 will typically be considered a single-use device.

In practice, for example, a surgeon will insert the flow device 140 into the operative member 120 of the hysteroscope 102 prior to introduction into the uterus of a patient. After hysteroscope introduction into the uterus, an inflow valve 146 of the flow device 140 will be opened and the uterus will be distended. Then, by opening an outflow valve 148 of the flow device 140, irrigation is achieved. In the case of a diagnostic procedure, the flow device 140 could stay in place for the duration of the surgery. In the case of an operative procedure, the flow device 140 is removed and an operative tool (e.g., the morcellator 112) is inserted into the scope 118.

While the best modes for carrying out the present invention have been described in detail, those familiar with the art to which this invention relates will recognize various alternative designs and embodiments for practicing the invention within the scope of the appended claims. For example, the sheath 104, the scope 118, and the surgical tool 122 can be circular, oval, or any other smooth shape (i.e., an unobtrusive shape such as a shape that does not have a outward extending flange). In another example, the operative member 120 can have a circular shape or any other similar shape to the illustrated D-shape.

1. (canceled)

2. A hysteroscopy system, comprising:
a proximal housing;
an elongated tubular scope extending distally from the proximal housing and having an internal lumen extending longitudinally through the elongated tubular scope;
a wall extending across the elongated tubular scope to divide the internal lumen into a first portion having a first cross-sectional area and a second portion having a second cross-sectional area and fluidly isolated from the first portion, wherein the first portion defines an operative channel that fully occupies the first cross-sectional area; and
a visualization device disposed within the second portion of the internal lumen, the visualization device occupying a cross-sectional area less than the second cross-sectional area such that the second cross-sectional area includes free space that is not occupied by the visualization device,
wherein the proximal housing and the operative channel are configured to receive a surgical tool inserted from a proximal end of the proximal housing through the proximal housing and through the operative channel.

3. The hysteroscopy system according to claim 2, wherein the first cross-sectional area is different from the second cross-sectional area.

4. The hysteroscopy system according to claim 2, wherein the first cross-sectional area is larger than the second cross-sectional area.

5. The hysteroscopy system according to claim 2, further comprising the surgical tool.

6. The hysteroscopy system according to claim 5, wherein the surgical tool is a morcellator.

7. The hysteroscopy system according to claim 2, wherein the visualization device includes fiber optics for illumination.

8. The hysteroscopy system according to claim 2, further comprising a valve coupled to the proximal housing and disposed in fluid communication with the internal lumen of the elongated tubular scope.

9. The hysteroscopy system according to claim 2, wherein the elongated tubular scope is configured for simultaneous inflow and outflow of fluid through the internal lumen of the elongated tubular scope.

10. The hysteroscopy system according to claim 2, wherein the proximal housing and the operative channel are capable of receiving a surgical tool having a diameter of 2.9 mm.

11. The hysteroscopy system according to claim 2, wherein the free space includes a first free space portion and a second free space portion separate from the first free space portion.

12. A hysteroscopy system, comprising:
a proximal housing;
an elongated tubular scope extending distally from the proximal housing and having an internal lumen extending longitudinally through the elongated tubular scope;
a wall extending across the elongated tubular scope to divide the internal lumen into a first portion having a first cross-sectional area and a second portion having a second cross-sectional area and fluidly isolated from the first portion, wherein the first portion defines an operative channel that fully occupies the first cross-sectional area; and
a visualization device disposed within the second portion of the internal lumen, the visualization device occupying a cross-sectional area less than the second cross-sectional area such that the second cross-sectional area includes first and second free space portions that are not occupied by the visualization device, wherein the first free space portion is separate from the second free space portion,
wherein the proximal housing and the operative channel are configured to receive a surgical tool inserted from a proximal end of the proximal housing through the proximal housing and through the operative channel.

13. The hysteroscopy system according to claim 12, wherein the first cross-sectional area is different from the second cross-sectional area.

14. The hysteroscopy system according to claim 12, wherein the first cross-sectional area is larger than the second cross-sectional area.

15. The hysteroscopy system according to claim 12, further comprising the surgical tool.

16. The hysteroscopy system according to claim 15, wherein the surgical tool is a morcellator.

17. The hysteroscopy system according to claim 12, wherein the visualization device is configured for illumination and image transmission.

18. The hysteroscopy system according to claim 12, wherein the visualization device includes fiber optics for illumination.

19. The hysteroscopy system according to claim 12, further comprising a valve coupled to the proximal housing and disposed in fluid communication with the internal lumen of the elongated tubular scope.

20. The hysteroscopy system according to claim 12, wherein the elongated tubular scope is configured for simultaneous inflow and outflow of fluid through the internal lumen of the elongated tubular scope.

21. The hysteroscopy system according to claim 12, wherein the proximal housing and the operative channel are capable of receiving a surgical tool having a diameter of 2.9 mm.
